(19) United States
(12) Patent Application Publication
(10) Pub. No.: US 2007/0197927 A1
(43) Pub. Date: Aug. 23, 2007

(54) Method for detecting cardiovascular problems using micro or nano vibrations

(75) Inventor: Eran Ofek, Bnei Brak (IL)

Correspondence Address: EDWARD S. SHERMAN, ESQ., 3554 ROUND BARN BLVD., SUITE 303, SANTA ROSA, CA, 95403, US

(73) Assignee: PHYSICAL LOGIC AG, Zug (CH)

(21) Appl. No.: 11/613,649

(22) Filed: Dec. 20, 2006

Related U.S. Application Data
(60) Provisional application No. 60/753,690, filed on Dec. 22, 2005.

(51) Int. Cl. A61B 5/04

(52) U.S. Cl. 600/514

(57) ABSTRACT

A method and apparatus for characterizing cardiac function and detecting disorders thereof deploys a plurality of sensors on skin surface or close to it, to acquire data for detecting, locating, analyzing and displaying the time variant shifts in the local center of gravity of the heart tissues and fluids. The method deploys a sensor apparatus that is able to detect micro and/or nano vibrations arising from characteristic events in the cardiac cycle and/or movement of the heart tissue and fluids. The time variant shifts of the heart's center of gravity may be displayed with other temporal measures of cardiac activity such an ECG to facilitate the detection and diagnosis of abnormalities, and/or confirm a diagnosis from the ECG.

### CROSS REFERENCE TO RELATED APPLICATIONS

present application claims priority to the U.S. provisional application, having Ser. No. 60/753,690 entitled “A method for detecting cardiovascular problems using micro or nano vibrations”, filed on Dec. 22, 2005, which is incorporated herein by reference.

### BACKGROUND OF INVENTION

The present invention relates to an apparatus and method for detecting, analyzing and displaying information indicative of a patient's cardiac function.

Numerous methods of measuring the quality and efficiency of a patient's cardiac function have been developed over the history of modern medicine, such as Electrocardiography (ECG), Ballistacardiography, Coronary Angiography, Positron Emission Tomography (PET), and Nuclear Cardiology. These methods either indirectly characterize the hearts'condition by its electric activity, momentum transfers or attempt to image at least portions of the heart.

Detection of body micro-vibration is known in the art, see for example R.

Strum, R, B. Nigg and E. A. Koller, “The impact of cardiac activity on triaxially recorded endogenous micro-vibrations of the body”, European Journal of Applied Physiology, vol. 44, pp. 83-96, 1980. Strum et al. evaluated the relationship between the cardiac activity and the micro-vibrations of the body and concluded that the most important source of whole-body micro vibrations is the cardiac activity.

Further, in U.S. Pat. No. 6,328,698 (to Matsumoto and issued Dec. 11, 2001), which is incorporated herein by reference, there is disclosed a diagnostic system and method for coronary artery disease which is operative to detect vibration signal of murmur deriving from the early stages of stenosis of coronary arteries. The vibration signals are detected using one or a plurality of laser source head and vibration detective sensor with laser displacement gauge and three-axial accelerometer, and the detector of vibration signal of environmental noise has three-axial accelerometer and supersensitive microphone.

While some of these methods can very accurately detect the presence of congenital or degenerative disorders very effectively, they generally require to varying degrees either complex equipment that prevents the patient from undergoing normal activity or the invasive or external attachment of sensitive leads making them unsuitable for continuous monitoring.

It is therefore a first object of the present invention to provide an improved method of measuring a patient's cardiac function that is non-invasive and can be easily attached to the patient for continuous use, if desired.

It is yet another objective of the invention to provide such a method that can provide superior and/or complimentary information to other cardiac diagnostic procedures that may be accomplished simultaneously.

### SUMMARY OF INVENTION

The present invention discloses a method to analyze the micro vibrations generated from the beating of the heart and detect various cardiovascular problems. A plurality of sensors is positioned in specific desirable locations on the person's body where preferably at least one is located in the general area of the person's chest. These sensors sense the micro-vibrations generated from the heart muscle contractions, valve opening/closing, and/or acceleration/de-acceleration of blood flow. The sensor signals are then filtered from noises in order to analyze the filtered signal for various cardiovascular problems. Once the method detects a specific problem, it will alert the person and/or physician. This method will improve the current methods of monitoring people at risk for various cardiovascular problems, potentially providing an “early warning system” for people at high risk to suffer from SCD (Sudden Cardiac Death).

In the present invention, the one object is achieved by an apparatus that includes a plurality of vibration detectors attached to the patient in communication with an optional computational unit. The computational unit deploys an algorithm to process the signals and calculate the temporal change in the 3-dimensional displacement of the center of gravity of the heart.

Another aspect of the invention is characterized by the process of placing at least one primary sensors on the patients skin in proximity to the heart, and then preferably but optionally placing at least one secondary sensor on the patients skin, receiving signals from the sensors, processing and filtering the signals to eliminate noise and then displaying the filtered signal as a 3-dimensional graph of the displacement of the center of gravity of the heart.

The above and other objects, effects, features, and advantages of the present invention will become more apparent from the following description of the embodiments thereof taken in conjunction with the accompanying drawings.

### BRIEF DESCRIPTION OF DRAWINGS

FIG. 1 is a schematic illustration of a patient being diagnosed with the inventive apparatus.

FIG. 2 is a flow chart illustrating the data collection and analysis of the signals obtained from the sensor shown in FIG. 1.

FIG. 3 is a theoretical example of the graphic output resulted from the method shown in FIG. 2.

### DETAILED DESCRIPTION

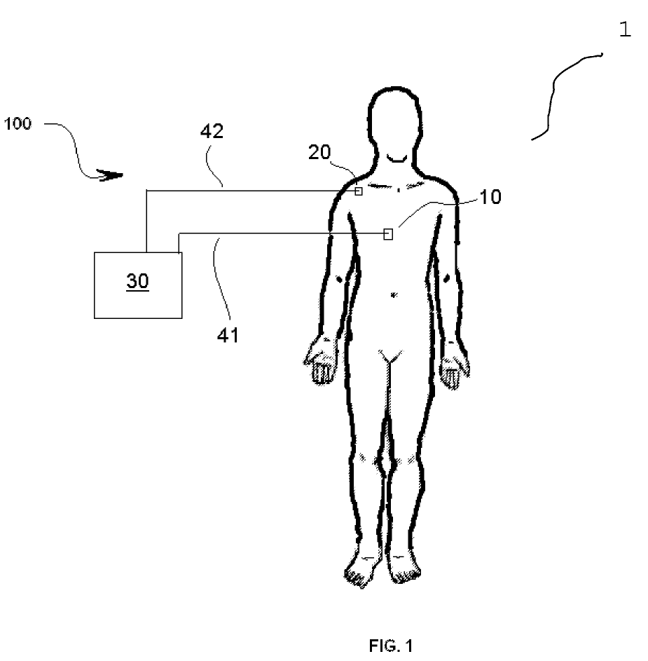

Referring to FIGS. 1 through 3, wherein like reference numerals refer to like components in the various views, there is illustrated therein a new and improved apparatus for cardiac diagnostics, generally denominated 100 herein.

First, it should be appreciated that the heart is a complex organ both physiologically and structurally. On a structure level, the heart is a two-stage pump with four chambers and two pairs of valves. The inventors have realized that the heart physiological characteristics may be diagnosed, in addition to traditional methods, by observing the structural variation of the heart during the cardiac cycle. Unlike most mechanical pumps, the heart itself undergoes changes in external shape due to both the contraction of cardiac muscle as well as the flow of blood into and out of the chambers. The inventor's have further appreciated that due to the inherent electromechanical coupling of the hearts function, as well as the elastic nature of both muscle and vascular tissue, the pumping action will generate numerous vibrations that propagate in multiple directions, eventually reaching the patients skin.

In accordance with the present invention, such vibrations are detected, analyzed and displayed to provide a measure of cardiac function that enables the detection and diagnosis of abnormalities. In such methods, as shown in FIG. 1, the patient 1 preferably has at least two sensors, 10 and 20, placed on the skin. The primary sensor 10 is placed near the heart. A secondary sensor 20 is placed more distal from the heart. Each sensor detects vibration arising from physiological functions. The primary sensor is placed closer to the heart to detect vibrations arising from the movement of the heart and the blood being pumped therein in the cardiac cycle. The signals from the sensors are received by an optional processing unit 30.

It should be appreciated by those skilled in the art, that the sensors 10, 20 and any other sensors can be physically on the patient's skin, near the skin or implanted within the patient (under the skin or anywhere else as known in the art)

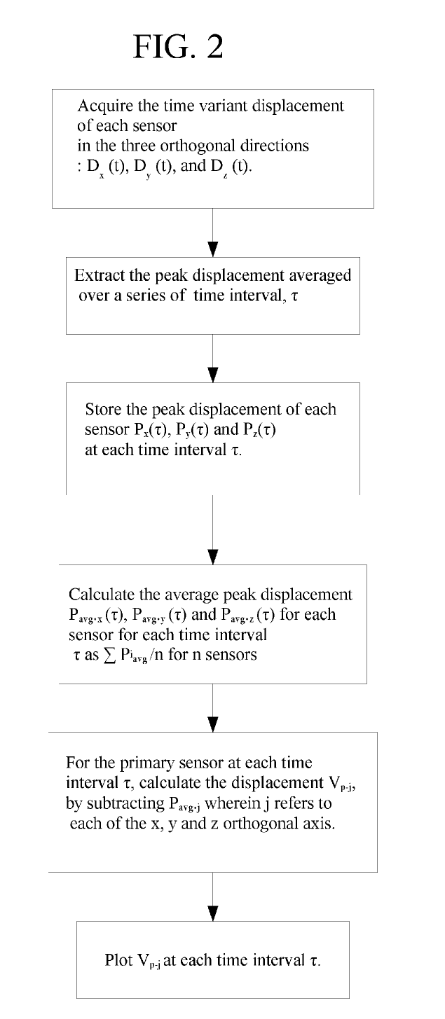

Each sensor is capable of measuring vibrations in three orthogonal directions. When the output of the first or primary sensor is suitably filtered to remove noise and vibrations not associated with the cardiac cycle, the amplitude of the remaining vibrations represents the movement of the heart in the three directions. Such filtering, and other computations are performed by the processing unit 30. It should be appreciated that the processing unit is preferably integrated into at least one of the sensors so that it can be worn by the patient during exercise or normal use, as well as when the patient is in a prone position and connected to the processing unit 30 by cables 41 and 42. It is preferable to use a plurality of sensors around the heart to more accurately separate and filter vibrations not associated with the motion of the heart. The output of each sensor can be compared with the average output of every other sensor, wherein the average output is filtered out as background noise. In this manner vibrations arising from the more remote sensors not associated with the hearts motion will be removed. The basic analysis algorithm is further explained with reference to FIG. 2.

As shown in the flow chart of FIG. 2, in the first step in the process 201 the sensors acquire the time variant displacement of each sensor in the three orthogonal directions: Dₓ (t), D_{y} (t), and D_{z} (t). In the next step in the process, 202, the peak displacement of the vibration sensor, that is the amplitude of the vibration, is extracted as the average over a series of time interval τ.

Preferably the cardiac cycle is divided into a sufficient number of time intervals to fully resolve each critical operative stage of the cardiac activity. In the next step in the process, 203, the peak displacement of each sensor Pₓ(τ), P_{y}(τ) and P_{z}(τ) at each time interval τ are stored for further calculation. However, such storage can be merely transitory for a very brief time period for continuous calculation in step 204. In step 204 the average peak displacement P_{avg·x} (τ), P_{avg·y} (τ) and P_{avg·z} (τ) for each sensor for each time interval τ is calculated as Σ Pⁱ_{avg}/n for n sensors. In the next step in the process, 205, for the primary sensor at each time interval τ, the displacement Vₚ₋ⱼ, is calculated by subtracting P_{avg·j} wherein j refers to each of the x, y and z orthogonal axis. The next, and potentially final step in one aspect of the invention is 206 is which Vₚ₋ⱼ is plotted at each time interval τ.

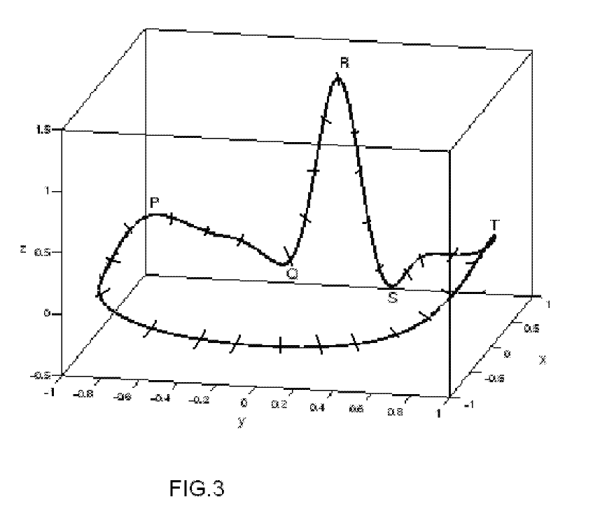

The resultant peak displacements, Vₚ₋ⱼ, in each of the x, y and z directions may be stored in a data structure for each time interval τ for displaying the displacements as a function of time. This peak amplitude in each cardiac cycle is then plotted as shown in the 3-dimensional graph of FIG. 3, or a beat spectrograph. The axes in the graph represent the three orthogonal dimensional coordinates. As the graph is theoretical, the magnitude of the displacement is relative for illustrative purposes. Thus, each cardiac cycle will generally be represented by a closed loop, as the heart returns to its original position at each cycle. Units of equal time are denoted by hash marks crossing the closed loop.

Further, as it is anticipated that via electromechanical coupling, the physical movement of heart muscle mass will correlate with electrical activity associated with one or more of the PQRS and T waves of ECG, the expected shape at these portions of the cardiac cycle are also indicated on the Figure. U.S. Pat. No. 5,554,177 (to Kieval for a “Method and apparatus to optimize pacing based on the intensity of acoustic signal” and issued Sep. 10, 1996), which is incorporated herein by reference, illustrates the general correlation of gross audio frequency vibrations with the electrical activity recorded by ECG, as well as other cardiac activity detectable by Doppler methods. Thus, it is expected that analysis of this graph can detect among the actual heart rate, various cardiovascular problems, examples of such problems can include: various cardiac arrhythmias, irregularities in blood flow to the heart, fibrillations, fluttering etc.

In other aspects of the invention, the displacements may used to derive a time and spatial correlation of the characteristic signals associated with a physical location within the heart. This correlation can be made by measuring the relative time it takes for a characteristic vibration mode to reach each sensor, and then triangulating a 3-dimensional position.

Preferably, the sensors are nano-sensors or other sensors of sufficiently small size so that they can be worn indefinitely on the patients' skin, or otherwise deployed in physiological communication with the patient, to optionally provide continuous measurement. U.S. Pat. No. 6,118,208 (which issued to Green, et al., Sep. 12, 2000 and is incorporated herein by reference) discloses an acoustic or vibration sensor particularly useful in detecting nano-vibrations. Other suitable sensors include, without limitation, accelerometers, hydrophones, microphones, laser velocimeters, strain gages, and motion detectors.

In other embodiments of the invention, the algorithm of FIG. 2 is operative to shift the Z-axis (or another axis that generally has the least displacement) of the displayed data for each cardiac cycle. Such periodic shifting will create a geometrical “spiral like” graph. In yet further embodiments of the invention include superimposing electrocardiograms on the beat spectrograph. In other embodiments of the invention, at least one sensor can be internal, implanted in or around the heart such as on a cardiac pacemaker or defibrillator device lead.

It is anticipated that the analysis of the graph in FIG. 3, and the variants thereon described above, will enable the clinician to detect various cardiovascular problems, such as arrhythmias, irregularities in blood flow to the heart etc. The method is non-invasive and sensitive to small changes unlikely to be revealed by examination with a stethoscope. Unlike the use of a stethoscope, the method permits quantitative diagnosis through comparison with standards and trend analysis.

While the invention has been described in connection with a preferred embodiment, it is not intended to limit the scope of the invention to the particular form set forth, but on the contrary, it is intended to cover such alternatives, modifications, and equivalents as may be within the spirit and scope of the invention as defined by the appended claims.

1. A method for measuring the cardiac function of a patient, the method comprising the steps of:
a) placing a sensor for detecting vibrations on at least on of the patients' skin, close to the patients' skin, under the patients' skin and implanted within the patient,
b) receiving signals from said sensor,
c) filtering the signals to eliminate noise and amplify relevant information,
d) processing the signals to determine the mean displacement of the heart in three orthogonal directions as a function of time or heartbeat cycle process duration.

2. A method for measuring the cardiac function of a patient according to claim 1, the method further comprising the steps of:
a) placing at least one secondary sensor for detecting vibrations on the patients skin,
b) receiving signals from said sensors,
c) filtering the signals to eliminate noise and amplify relevant information,
d) processing the signals to determine the mean displacements in three orthogonal directions as a function of time or heartbeat cycle process duration.

3. A method for measuring the cardiac function of a patient according to claim 1, the method further comprising the steps of correlating the time and spatial location of the characteristic signals with a physical location within the heart.

4. A computer or other device readable medium having stored thereon a data structure comprising:
a) a first data field for storing the time of a cardiac event,
b) a second data field for storing the resolved spatial coordinates of the event for each record in the first data fields,
c) wherein the resolved spatial coordinates are relative to the center of gravity of the heart.

5. An apparatus for measuring the cardiac function of a patient, comprising
a) one or more sensors for detecting vibrations,
b) a signal processing and computational unit for receiving the output of said sensors, the computation unit being operative to:
i) receive signals from said one or more sensors,
ii) filter the signals to eliminate noise and amplify relevant data,
iii) process the filtered signals to detect characteristic signals, iv) store the time and spatial location of the characteristic signals in a data structure for further processing and display.

6. An apparatus for measuring the cardiac function of a patient according to claim 5 further operative to process the filtered signals in real-time.

7. An apparatus for measuring the cardiac function of a patient according to claim 5 portion operative to process the filtered signals in real time does not require storage.
